(19) United States
(12) Patent Application Publication
(10) Pub. No.: US 2020/0121522 A1
(43) Pub. Date: Apr. 23, 2020

(54) ABSORBENT ARTICLE WITH AEROGEL ABSORBENT

(71) Applicant: The Procter & Gamble Company, Cincinnati, OH (US)

(72) Inventor: Vito CARLA, Cincinnati, OH (US)

(21) Appl. No.: 16/655,752

(22) Filed: Oct. 17, 2019

Related U.S. Application Data
(60) Provisional application No. 62/747,414, filed on Oct. 18, 2018.

(51) Int. Cl. A61F 13/53; A61F 13/511; A61F 13/514

(52) U.S. Cl. CPC A61F 13/53; A61F 13/511; A61F 2013/15544; A61F 2013/530613; A61F 2013/530051; A61F 13/514

(57) ABSTRACT

A disposable absorbent article is disclosed. The article may include a liquid permeable topsheet, a liquid-impermeable backsheet, and an absorbent core structure disposed between the topsheet and the backsheet, wherein the absorbent core structure includes a cellulose aerogel body.

### BACKGROUND OF THE INVENTION

Wearable, disposable absorbent articles such as feminine hygiene pads, incontinence pads, disposable incontinence pants, disposable diapers, disposable training pants and wound dressing/bandaging products typically include an absorbent structure including one or more layers formed of absorbent material adapted to accept, distribute, absorb and store a quantity of typically aqueous exudate/bodily fluids such that they are removed from contact with the wearer's skin surfaces but prevented from escaping so as to soil, e.g., undergarments, outer clothing or bedclothes. A continuing concern related but not limited to feminine hygiene pads (menstrual pads) is a consumer desire for the product to be light, thin/non-bulky and flexible so as to be discreet when worn under clothing, comfortable, and accommodating of the wearer's ordinary movements throughout her day, while still providing adequate absorption capability and security against leakage.

Although materials and structures have been developed to date that are sufficiently effective at fluid handling and storage for purposes of manufacturing commercially acceptable absorbent articles, room for improvements always remains. Any improvements in the combination of features including material cost, processability/manufacturing efficiency, mechanical robustness, weight, bulk, flexibility/pliability and/or fluid handling characteristics will always be welcome potential contributors to improvements to absorbent structures, within absorbent articles in which they are included.

### DESCRIPTION OF THE DRAWINGS

FIG. 1 is a plan view of an example of an absorbent article in the form of a feminine hygiene pad, shown with the wearer-facing surface facing the viewer, and a partial cutaway of the topsheet component to shown underlying components.

FIG. 2 is a schematic lateral cross section of the components of the article shown in FIG. 1, taken along line 2-2 in FIG. 1.

FIG. 3 is a enlarged perspective schematic illustration view of a portion of a cellulose nanofibril aerogel structure.

FIG. 4 is a schematic perspective illustration depicting positioning of an absorbent core structure component formed of a cellulose nanofibril aerogel, between overlying and underlying web components.

### DESCRIPTION OF EMBODIMENTS

### Definitions

Herein, the term “absorbent article” is used generally to refer to products such as durable or disposable feminine hygiene pads (including menstrual pads and panty liners), incontinence pads, incontinence pants, diapers, training pants, nursing pads, bandages and wound dressings, which are applied to and worn over the body to receive, absorb and contain discharged bodily fluids. Such products typically include an absorbent core structure formed of one or more materials selected and configured to receive, accept, distribute or transport, absorb, retain and store discharged fluids (e.g., menses, breast milk, urine, liquidous stool, wound discharge, etc.) until such time as the product is replaced by a fresh product, or is no longer needed.

Herein, “aerogel” refers to a synthetic, porous material derived from a gel, in which the liquid volume component of the gel has been substantially removed and replaced with a gas.

With respect to a component of an absorbent article, “liquid impermeable” means substantially resistive to through-penetration of liquid water and bodily fluids including urine and menses, at room temperature and ordinary conditions of use of the article.

With respect to a component of an absorbent article, “liquid permeable” means substantially permitting of through-penetration of liquid water and urine at room temperature and ordinary conditions of use of the article.

“x-y plane”—with respect to a web or layer component of an absorbent article, refers to the plane substantially occupied or defined by a major surface of the web or layer component when laid out flat.

“z direction”—with respect to a web or layer component of an absorbent article, refers to a direction orthogonal to the plane substantially occupied or defined by a major surface of the web or layer component when laid out flat.

### Feminine Hygiene Pads

Generally

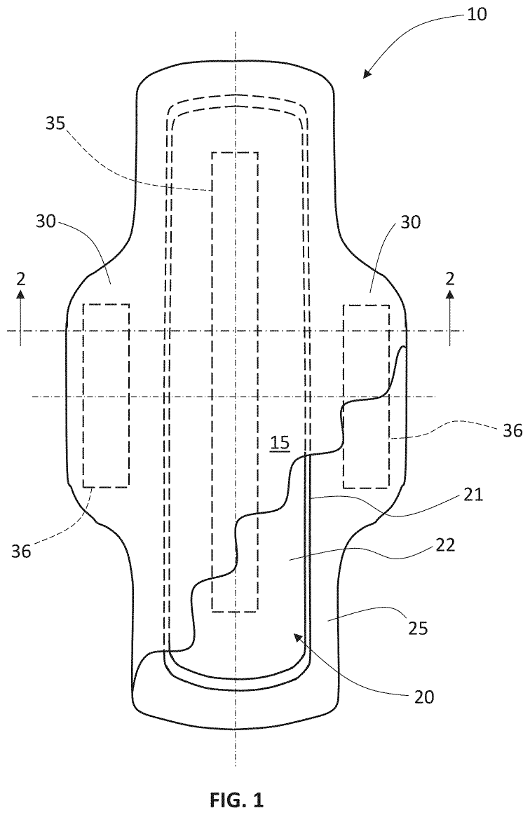

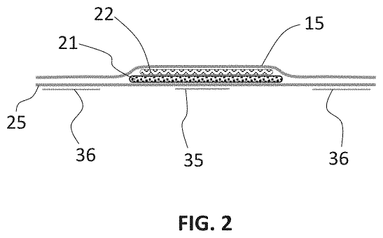

Referring to FIGS. 1 and 2, a non-limiting example of the types of absorbent articles contemplated by the present disclosure is a disposable feminine hygiene pad 10 (also known as a menstrual pad or catamenial pad). A feminine hygiene pad in one of its more simple forms includes components similar to those found in more complex products such as disposable incontinence pads, disposable diapers, incontinence pants and training pants. Referring to the FIG. 1, a typical feminine hygiene pad 10 may include a liquid permeable, wearer-facing topsheet 15, a liquid impermeable, outward-facing backsheet 25 and an absorbent core structure 20 disposed between the topsheet and the backsheet, within an enveloping structure formed thereby. The topsheet and the backsheet may be bonded or adhered to each other about their respective perimeters to envelope and contain the absorbent core structure. The pad may include a main deposit of adhesive 35 that the wearer/user may use to affix the pad to the inside surface of the wearer's underpants, to hold the pad in place. The pad may be provided with a section of removable release tape or paper over the adhesive to protect the adhesive from contact with other surfaces until the time the pad is to be used.

An absorbent core structure 20 may be formed of one or more layers, and may include an absorption layer 21 and an acquisition/distribution layer 22, sometimes also known as a “secondary topsheet”. The acquisition/distribution layer 22 may be disposed closer to the topsheet (and thereby closer to the wearer and location of discharge of fluids) and may be formed of a nonwoven web or batt of fibers or filaments. The acquisition/distribution layer 22 may have higher permeability, lower capillarity and lower absorption capacity relative those same properties of the absorption layer 21. The absorption layer 21 may be disposed beneath the acquisition/distribution layer 22 (i.e., closer to the backsheet 25), and may be formed of one or more layers of an absorbent material or physical blend of particles and/or fibers of one or more absorbent materials. The absorption layer 21 may be configured to have generally lower permeability but higher capillarity and higher absorption capacity relative those same properties of the acquisition/distribution layer 22.

Thus, in typical configurations, the acquisition/distribution layer 22 serves to receive and rapidly distribute fluids in directions along the x-y plane of the topsheet at the time of discharge, and the absorption layer 21 serves to draw the fluids from the acquisition/distribution layer in a z-direction, and retainably absorb them at location removed from the wearer's skin but isolated from the wearer's underpants, outer garments, bedclothes, etc. by the liquid impermeable backsheet 25.

A feminine hygiene pad 10 also may include laterally extending portions of the topsheet and/or backsheet sometimes known as “wings” 30. Wings 30 may be included so as to provide extra material to wrap over and about inside portions of leg edges of the wearer's underpants in the crotch region thereof. Wings may be provided with side deposits of adhesive 36, e.g., as shown, that the wearer/user may use to affix the wings, wrapped about the inside portions of the underpants' leg edges, to the outer surfaces of the underpants in the crotch region. Wings may serve to provide an alternative or supplementary way to hold the pad in place within the user/wearer's underpants during use/wear, and may also serve to provide added protection against soiling of the underpants about the leg edges, by discharged fluid that has not been absorbed. The pad may be provided with sections of removable release tape or paper over the side deposits of adhesive, to protect the adhesive from contact with other surfaces until the time the pad is to be used.

The pad 10 may include other features known in the art to enhance its consumer appeal, comfort and performance, including but not limited to odor control technologies (odor neutralization and/or masking); lotions applied to skin-contacting surfaces (for skin protection and/or comfort); channels in the topsheet and/or absorbent core structure (to enhance flexibility and/or to facilitate fluid distribution along/across the absorbent core structure) and embossed, impressed or imprinted features and designs for functional or esthetic purposes.

Topsheet

The topsheet 15 may be formed of any suitable material that is liquid permeable and compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, also provides for the transfer or migration of the lotion composition onto an external or internal portion of a wearer's skin. A suitable topsheet may be made of various materials such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims, or combinations thereof.

Apertured film materials suitable for use as the topsheet include those apertured plastic films that are non-absorbent and pervious to body exudates and provide for minimal or no flow back of fluids through the topsheet. Nonlimiting examples of other suitable formed films, including apertured and non-apertured formed films, are more fully described in U.S. Pat. Nos. 3,929,135; 4,324,246; 4,342,314; 4,463,045; 5,006,394; 4,609,518, and 4,629,643. Commercially available formed filmed topsheets include those topsheet materials on feminine hygiene pad products marketed by the Procter & Gamble Company (Cincinnati, Ohio) under the trade name or trademark DRI-WEAVE.

Non-limiting examples of woven and nonwoven materials suitable for use as the topsheet include fibrous materials made from natural fibers, modified natural fibers, synthetic fibers, or combinations thereof. These fibrous materials may be either hydrophilic or hydrophobic, but it is preferable that the topsheet be hydrophobic or rendered hydrophobic. As an option portions of the topsheet may be rendered hydrophilic, by the use of any known method for making topsheets containing hydrophilic components. One such method include treating an apertured film component of a nonwoven/apertured thermoplastic formed film topsheet with a surfactant as described in U.S. Pat. No. 4,950,264. Other suitable methods describing a process for treating the topsheet with a surfactant are disclosed in U.S. Pat. Nos. 4,988,344 and 4,988,345. The topsheet may have hydrophilic fibers, hydrophobic fibers, or combinations thereof.

A particularly preferred topsheet comprises staple length polypropylene fibers having a denier of about 1.5, such as Hercules type 151 polypropylene marketed by Hercules, Inc. of Wilmington, Del. As used herein, the term “staple length fibers” refers to those fibers having a length of at least about 15.9 mm (0.62 inches).

When the topsheet comprises a nonwoven fibrous material in the form of a nonwoven web, the nonwoven web may be produced by any known procedure for making nonwoven webs, nonlimiting examples of which include spunbonding, carding, wet-laid, air-laid, meltblown, needle-punching, mechanical entangling, thermo-mechanical entangling, and hydroentangling. A specific example of a suitable meltblown process is disclosed in U.S. Pat. No. 3,978,185. The nonwoven may be compression resistant as described in U.S. Pat. No. 7,785,690. The nonwoven web may have loops as described in U.S. Pat. No. 7,838,099.

Other suitable nonwoven materials include low basis weight nonwovens, that is, nonwovens having a basis weight of from about 18 g/m² to about 25 g/m². An example of such a nonwoven material is commercially available under the tradename P-8 from Veratec, Incorporation, a division of the International Paper Company located in Walpole, Mass. Other nonwovens are described in U.S. Pat. Nos. 5,792,404 and 5,665,452.

The topsheet may comprise tufts as described in U.S. Pat. Nos. 8,728,049; 7,553,532; 7,172,801, or 8,440,286. The topsheet may have an inverse textured web as described in U.S. Pat. No. 7,648,752. Tufts are also described in U.S. Pat. No. 7,410,683.

The topsheet may have a pattern of discrete hair-like fibrils as described in U.S. Pat. No. 7,655,176 or 7,402,723.

The topsheet may comprise one or more structurally modified zones as described in U.S. Pat. No. 8,614,365. The topsheet may have one or more out of plane deformations as described in U.S. Pat. No. 8,704,036. The topsheet may have a masking composition as described in U.S. Pat. No. 6,025,535.

Backsheet

The backsheet 25 is typically included to act as a barrier to passage of bodily fluids to the wearer's underpants, outer garments, bedclothes, etc. Further, the barrier properties of the backsheet permit manual removal, if a wearer so desires, of the article from the wearer and/or the wearer's underpants, with reduced risk of hand soiling. The backsheet 25 is preferably formed of material that is soft, smooth, compliant, and liquid impermeable, to provide for softness and conformability for comfort, and is quiet in that it does not emit unwanted levels of noise (e.g., “crinkling” sounds) when it is folded, gathered, bent, twisted, etc.

The backsheet 25 may be formed of a wet laid fibrous assembly having a temporary wet strength resin incorporated therein as described in U.S. Pat. No. 5,885,265. The backsheet may further be coated with a water resistant resinous material that causes the backsheet to become impervious to bodily fluids without impairing the spreading of adhesive materials thereon.

Another suitable backsheet material is a polyethylene film having a thickness of from about 0.012 mm (0.5 mil) to about 0.051 mm (2.0 mils). The backsheet may be embossed and/or matte finished to provide a more clothlike appearance. Further, the backsheet may be manufactured or processed so as to be permit vapors to escape from the absorbent core 42 (i.e., the backsheet is breathable) while still preventing body fluids from passing through the backsheet. A preferred microporous polyethylene film which is available from Tredegar Corporation, Virginia, USA, under Code No. XBF-1 12W.

For a stretchable but non-elastic backsheet, one material may be used is a hydrophobic, stretchable, spun laced, non-woven material having a basis weight of from about 30 to 40 g/m², formed of polyethylene terephthalate or polypropylene fibres. This material is breathable, i.e. permeable to water vapour and other gases.

For an elastic backsheet, one material which may be used is an elastic film sold under the trade mark EXX500 by Exxon Corporation. The material of this film is formed from an elastomeric base composition consisting of a styrene block copolymer. However, this material is not breathable. Another material which may be used for an elastic backsheet is a plastic film that has been subjected to a process that provides it with elastic-like properties without attaching elastic strands to the film, and may for example comprise a formed film made in accordance with U.S. Pat. Nos. 4,342,314 and 4,463,045.

Suitable breathable backsheets for use herein include all breathable backsheets known in the art. In principle there are two types of breathable backsheets, single layer breathable backsheets which are breathable and impervious to liquids and backsheets having at least two layers, which in combination provide both breathability and liquid imperviousness. Suitable single layer breathable backsheets for use herein include those described for example in GB A 2184 389; GB A 2184 390; GB A 2184 391; U.S. Pat. Nos. 4,591,523; 3,989,867; 3,156,242, and WO 97/24097.

The backsheet may include two or more layers. In some examples, a first layer may be formed of a vapor and air permeable apertured, formed film, and a second layer may be formed of a breathable microporous film layer, as described in U.S. Pat. No. 6,462,251. Other examples of dual- or multi-layer breathable backsheets that may be suitable for use herein include those described in U.S. Pat. Nos. 3,881,489; 4,341,216; 4,713,068; 4,818,600; EP 0 203 821; EP 0 710 471; EP 0 710 472, and EP 0 793 952.

The backsheet may be made to be vapor permeable as described in U.S. Pat. No. 6,623,464 or 6,664,439. The backsheet may be formed from any vapor permeable material known in the art. The backsheet may be formed of, or include, a microporous film, an apertured formed film, or other polymer film that is vapor permeable, or rendered to be vapor permeable, as is known in the art.

The backsheet may be formed of a nonwoven web having a basis weight between about 20 gsm and about 50 gsm. In one embodiment the backsheet is a relatively hydrophobic spunbond nonwoven web available from Fiberweb Neuberger, under the designation F102301001. The backsheet may be coated with a non-soluble, liquid swellable material as described in U.S. Pat. No. 6,436,508.

Absorbent Core Structure

The absorbent core may be any absorbent means capable of absorbing or retaining liquids discharged by the body (e.g., menstrual fluid and/or urine). The absorbent core structure may be manufactured in a wide variety of sizes and shapes (e.g., rectangular, oval, hourglass, dog bone, asymmetric, etc.) and from a wide variety of liquid-absorbent materials commonly used in feminine hygiene pads and other absorbent articles such as comminuted wood pulp which is generally referred to as airfelt. Examples of other suitable absorbent materials include creped cellulose wadding; meltblown polymers including coform; chemically stiffened, modified or cross-linked cellulosic fibers; synthetic fibers such as crimped polyester or polyolefin fibers; peat moss; tissue including tissue wraps and tissue laminates; absorbent foams; absorbent sponges; superabsorbent polymers; absorbent gelling materials, or any equivalent material or combinations of materials, or mixtures of these. The absorbent core structure may have more than one layer wherein each layer may be identical or distinct in one or more property or composition from another layer. A particularly preferred absorbent core structure is made of thermally bonded airlaid material having less than 50 percent synthetic fibers. Synthetic fibers are preferred due to the ease with which they fuse together to join the core and topsheet as described below. A particularly preferred synthetic fiber is a bi-component material having a polyethylene sheath and a polypropylene center.

The configuration and construction of the absorbent core structure may also be varied (e.g., the absorbent core structure may have varying caliper zones (e.g., profiled so as to be thicker in the center), hydrophilic gradients, superabsorbent gradients, or lower density and lower average basis weight acquisition zones, or may comprise one or more layers or structures). The total absorbent capacity of the absorbent core structure should, however, be compatible with the design loading and the intended use of the article. Further, the size and absorbent capacity of the absorbent core may be varied to accommodate different uses such as incontinence pads, panty liners, regular feminine hygiene pads, or overnight feminine hygiene pads.

The fluid absorbent material may include any of a variety of materials commonly used in disposable absorbent articles. Examples of suitable absorbent materials include creped cellulose wadding, cotton fluff, and citric acid cross-linked cellulose pulp disclosed in U.S. Pat. Nos. 5,190,563; 5,183,707, and 5,137,537; synthetic fibers disclosed in U.S. Pat. No. 4,578,414; absorbent foams, absorbent sponges, superabsorbent composites, superabsorbent foam, and super absorbent polymers. A preferred fluid absorbent material is comminuted and airlaid wood pulp fibers commonly referred to as absorbent fluff. An absorbent fluff having a density of from about 0.05 g/cm³ to about 0.175 g/cm³ is generally acceptable.

The absorbent core structure may comprise a substrate and superabsorbent polymer layer as those described in U.S. Pat. No. 8,124,827; U.S. patent application Ser. No. 12/718,244; U.S. patent application Ser. No. 12/754,935, or U.S. Pat. No. 8,674,169.

More preferred fluid absorbent materials are the absorbent gelling materials. As is well known in the art, fluid absorbent gelling materials (sometimes referred to as “AGM” or “superabsorbents”) are broadly used in fluid absorbent articles. In general, such AGM's have been used only for their fluid-absorbing properties. Such materials form hydrogels on contact with water (e.g., with urine, blood, and the like). One highly preferred type of hydrogel-forming, absorbent gelling material is based on the hydrolyzed polyacids, especially neutralized polyacrylic acid. Hydrogel-forming polymeric materials of this type are those which, upon contact with fluids (i.e., liquids) such as water or body fluids, imbibe such fluids and thereby form hydrogels. In this manner, fluid discharged into the fluid absorbent structures herein may be acquired and held. These preferred fluid absorbent gelling materials will generally comprise substantially water-insoluble, slightly cross-linked, partially neutralized, hydrogel-forming polymer materials prepared from polymerizable, unsaturated, acid-containing monomers. In such materials, the polymeric component formed from unsaturated, acid-containing monomers may comprise the entire gelling agent or may be grafted onto other types of polymer moieties such as starch or cellulose. The hydrolyzed polyacrylic acid grafted starch materials are of this latter type. Thus the preferred fluid absorbent gelling materials include hydrolyzed polyacrylonitrile grafted starch, hydrolyzed polyacrylate grafted starch, polyacrylates, maleic anhydride-iso-butylene copolymers and combinations thereof. Especially preferred fluid absorbent gelling materials are the hydrolyzed polyacrylates and hydrolyzed polyacrylate grafted starch.

Whatever the nature of the polymer components of the preferred fluid absorbent gelling materials, such materials will in general be slightly cross-linked. Cross-linking serves to render these preferred hydrogel-forming absorbent materials substantially water-insoluble, and cross-linking also in part determines the gel volume and extractable polymer characteristics of the hydrogels formed therefrom. Suitable cross-linking agents are well known in the art and include, for example: (1) compounds having at least two polymerizable double bonds; (2) compounds having at least one polymerizable double bond and at least one functional group reactive with the acid-containing monomer material; (3) compounds having at least two functional groups reactive with the acid-containing monomer material, and (4) polyvalent metal compounds which can form ionic cross-linkages. Preferred cross-linking agents are the di- or polyesters of unsaturated mono- or polycarboxylic acids with polyols, the bisacrylamides and the di- or triallyl amines. Especially preferred cross-linking agents are N,N′-methylenebisacrylamide, trimethylol propane triacrylate and triallyl amine. The cross-linking agent will generally comprise from about 0.001 mole percent to about 5 mole percent of the preferred materials. More preferably, the cross-linking agent will comprise from about 0.01 mole percent to about 3 mole percent of the absorbent gelling materials used herein.

The preferred, slightly cross-linked, hydrogel-forming absorbent gelling materials will generally be employed in their partially neutralized form. For purposes described herein, such materials are considered partially neutralized when at least about 25 mole percent, preferably at least about 50 mole percent, and more preferably at least about 75 mole percent, of monomers used to form the polymer are acid group-containing monomers which have been neutralized with a salt-forming cation. Suitable salt-forming cations include alkali metal, ammonium, substituted ammonium and amines. This percentage of the total monomers utilized which are neutralized acid group-containing monomers is referred to as the “degree of neutralization.” Typically, commercial fluid absorbent gelling materials have a degree of neutralization somewhat less than about 90%.

The preferred fluid absorbent gelling materials used herein are those which have a relatively high capacity for imbibing fluids encountered in the fluid absorbent articles; this capacity may be quantified by referencing the “gel volume” of said fluid absorbent gelling materials. Gel volume may be defined in terms of the amount of synthetic urine absorbed by any given fluid absorbent gelling agent buffer and is specified as grams of synthetic urine per gram of gelling agent.

Gel volume in synthetic urine (see Brandt et al., below) may be determined by forming a suspension of about 0.1-0.2 parts of dried fluid absorbent gelling material to be tested with about 20 parts of synthetic urine. This suspension is maintained at ambient temperature under gentle stirring for about 1 hour so that swelling equilibrium is attained. The gel volume (grams of synthetic urine per gram of fluid absorbent gelling material) is then calculated from the weight fraction of the gelling agent in the suspension and the ratio of the liquid volume excluded from the formed hydrogel to the total volume of the suspension. The preferred fluid absorbent gelling materials useful in this invention will have a gel volume of from about 20 to 70 grams, more preferably from about 30 to 60 grams, of synthetic urine per gram of absorbent gelling material.

The fluid absorbent gelling materials hereinbefore described are typically used in the form of discrete particles. Such fluid absorbent gelling materials may be of any desired shape, e.g., spherical or semi-spherical, cubic, rod-like polyhedral, etc. Shapes having a large greatest dimension/smallest dimension ratio, like needles and flakes, are also contemplated for use herein. Agglomerates of fluid absorbent gelling material particles may also be used.

The size of the fluid absorbent gelling material particles may vary over a wide range. For reasons of industrial hygiene, average particle sizes smaller than about 30 microns are less desirable. Particles having a smallest dimension larger than about 2 mm may also cause a feeling of grittiness in the absorbent article, which is undesirable from a consumer aesthetics standpoint. Furthermore, rate of fluid absorption may be affected by particle size. Larger particles have very much reduced rates of absorption. Fluid absorbent gelling material particles preferably have a particle size of from about 30 microns to about 2 mm for substantially all of the particles. “Particle Size” as used herein means the weighted average of the smallest dimension of the individual particles.

The amount of fluid absorbent gelling material particles used in absorption layers will depend upon the degree of fluid absorbent capacity desired, and will generally comprise from about 2% to about 50% by weight of the absorption layer, more typically from about 5% to about 20% by weight of the absorption layer.

When fluid absorbent gelling material particles are to be used in the cores of the fluid absorbent articles herein, such cores may be prepared by any process or technique which provides a web comprising a combination of the fibers and the gelling material particles. For example, web cores may be formed by air-laying a substantially dry mixture of hydrophilic fibers and fluid absorbent gelling material particles and, if desired or necessary, by densifying the resulting web. Such a procedure is described more fully in U.S. Pat. No. 4,610,678. As indicated in U.S. Pat. No. 4,610,678, the air-laid webs formed by such a procedure will preferably comprise substantially unbonded fibers and will preferably have a moisture content of about 10% or less.

Another example combining the fibers and the gelling material particles is a tissue laminate. Such an absorption layer is described more fully in U.S. Pat. Nos. 4,950,264; 5,009,653; WO 93/01785, and WO 93/01781, all of which are incorporated herein by reference. As indicated in these references, glue is applied to an air-laid, latex-bonded tissue and absorbent gelling material is added and then the tissue is folded over.

The density of an absorption layer that includes webs of hydrophilic fibers and fluid absorbent gelling material particles may be of importance in determining the fluid absorbent properties of the layer and of the articles in which such cores are employed. The density of such absorption layers herein will preferably be in the range of from about 0.06 to about 0.3 g/cm³, and more preferably within the range of from about 0.09 to about 0.22 g/cm³. Typically the basis weight of the absorption layers herein may range from about 0.02 to 0.12 g/cm².

Density values for cores of this type may be calculated from basis weight and caliper. Caliper is measured under a confining pressure of 0.137 psi (0.94 kPa). Density and basis weight values include the weight of the fluid absorbent gelling materials and the odor-control material. Density of the cores herein need not be uniform throughout the core. Within the density ranges set forth above, the cores may contain regions or zones of relatively higher or relatively lower density.

The size of the fluid absorbent element is dictated by the exact product design selected. The absorbent core may include other optional components. One such optional component is the core wrap, i.e., a material, typically but not always a nonwoven material, which either partially or totally surrounds the core. Suitable core wrap materials include, but are not limited to, cellulose, hydrophilically modified nonwoven materials, perforated films and combinations thereof.

It is contemplated herein that particles, bodies or structures formed of particular aerogels may be used to supplement, or substitute for, one or more absorbent materials currently used in the art as components of absorbent core structures and/or absorption layer components thereof, in absorbent articles.

Aerogels have been known for a number of years. An aerogel is a highly porous, extremely low-density solid made by removing the liquid component of a gel, via a process that leaves the solid matrix structure within the gel intact after the liquid is gone. Early aerogels were produced by drying silica hydrogels under supercritical conditions. The resulting product is a highly porous, ultralight, translucent material with substantial thermal insulating properties. The material is also naturally hydrophilic and absorbent. However, it is extremely brittle and fragile when dry, and its structure fails and collapses quickly under the weight of absorbed water. These characteristics make silica aerogels unsuitable for use as aqueous liquid absorbers.

Later-discovered aerogels have been produced of other materials such as carbon (graphene), and have proven to have a number of useful characteristics including mechanical robustness. However, these aerogels are not hydrophilic and are, therefore, not useful as aqueous liquid absorbers. Aerogels have also been produced of a number of other component materials, but for the reasons mentioned above or other reasons, have not be been deemed useful as absorbers of aqueous fluids, discouraging their consideration for this purpose.

Previously, it had been theorized that aerogels, xerogels or cryogels might be used as absorbent materials in absorbent articles. See U.S. Pat. No. 6,565,961. That reference, however, does not identify any particular type of aerogel as preferred or even effective, does not provide any examples, and does not provide any basis for any reasonable expectation of success. There is no indication in that reference that any materials identified therein could be successfully produced and included as absorbent materials in products of the type contemplated therein. It is believed aerogels known at the time of that publication lacked substantial physical robustness and mechanical strength in dry and/or wet states, tending to be brittle and easily crushed and pulverized when dry, and/or too deformable, collapsible and/or fluid when wetted, to be practical. Although an exhaustive search has not been performed, it is not believed that there was any indication in the art that such materials would be sufficient or effective for purposes of forming components of an absorbent core structure of an absorbent article. This is because such an application typically calls for materials that are sufficiently mechanically robust for purposes of manufacturing processes, shipping, handling and use, in addition to being hydrophilic and absorptive of aqueous liquids. Such components typically are required to substantially retain their shapes, sizes and positions, during manufacturing and handling of the product (i.e., when dry), and during wear and use of the product (including when wetted).

More recently, it has been discovered that aerogels may be produced from cellulose nanofibrils. It has been discovered further, that particular process steps may be employed to produce aerogels that have substantial mechanical robustness in both dry and wet conditions, and that such aerogels are hydrophilic and water-absorbent.

Cellulose nanofibrils may be obtained or derived from any suitable source. For example, cellulose nanofibrils may be obtained or derived from bleached wood pulps (e.g., eucalyptus, birch kraft pulp, softwood sulfite cellulose pulp); microcrystalline cellulose (MCC) or cellulose nanofiber (CNF) suspensions (purchased as such), discarded leaves (e.g., pineapple, licuri, banana, pine, pomelo, bamboo leaves), citrus waste, banana rachis, bamboo fibers, maize straw, corn husks, cotton linters, green algae (cladophora sp.), coir, kapok, sisal or even recycled cellulose fibers from paper waste. In some circumstances it may be desirable that the source of the cellulose nanofibrils be rice straw, due to abundant and widespread farming of rice as a food crop.

It is contemplated that the aerogels useful for purposes described herein may include one or more cellulose nanofibril aerogels more recently composed and manufactured as described in US 2019/0309144 and/or in any one or more of the following publications: Rajender R. Mallepally, Ian Bernard, Michael A. Main, Kevin R. Ward and Mark A. McHugh, Superabsorbent Alginate Aerogels, J. of Supercritical Fluids 79 (2013) 202-208 (Elsevier B.V. 2012) (“Mallepally et al.”); Feng Jiang and You-Lo Hsieh, Super Water Absorbing and Shape Memory Nanocellulose Aerogels from TEMPO-Oxidized Cellulose Nanofibrils via Cyclic Freezing-Thawing, 1 J. Mater. Chem. A, 2014, 2, 350 (Royal Society of Chemistry 2014) (“Jiang & Hsieh I”); Nathalie Lavoine and Lennart Bergstrom, Nanocellulose-Based Foams and Aerogels: Processing, Properties, and Applications, J. Mater. Chem. A, 2017, 5, 16015 (Royal Society of Chemistry 2017) (“Lavoine & Bergstrom”), and/or Feng Jian and You-Lo Hsieh, Cellulose Fiber Aerogels: Synergistic Improvement of Hydrophobicity, Strength, and Thermal Stability via Cross-Linking with Diisocyanate, ACS Appl. Mater. Interfaces 2017, 9, 2825-2834 (American Chemical Society 2-17) (“Jiang & Hsieh II”), which are incorporated by reference herein to the extent not inconsistent herewith. It is believed that such aerogels may be both biocompatible, and have substantially improved physical and mechanical robustness in both dry and wetted conditions, making them suitable for use in forming components of an absorbent core structure of an absorbent article.

It is contemplated that the one or more aerogels may be included as component(s) to supplement or substitute for currently used absorbent and/or liquid-handling materials including polymeric and/or cellulose fiber nonwoven web materials, batts, particles or fibers of synthetic materials known in the art as “superabsorbent polymers” (SAP) or “absorbent gelling materials”) (AGM), absorbent foams (including “high internal phase emulsion” (HIPE) foams), etc.).

It is further contemplated that a cellulose nanofibril aerogel described by one of the above-cited references might be manufactured to have a shaped volume via one of the techniques described. Thereafter, the shaped aerogel might be ground or otherwise relatively finely divided to produce aerogel particles. The aerogel particles might be incorporated as components in an absorbent core structure by blending them within a matrix of cellulose pulp fibers and depositing the blend onto a forming surface to form a batt of the combined/blended materials; by distributing and disposing them onto a substrate (e.g., a nonwoven web or polymeric film) with, for example, an adhesive, or by blending them with a binder and shaping the blend into any desired shape for a component of an absorbent core, such a shaped layer component such as currently found in marketed products having absorbent core components formed if HIPE foam, for example, ALWAYS INFINITY feminine hygiene pads marketed and sold by The Procter & Gamble Company, Cincinnati, Ohio.

It is further contemplated that a cellulose nanofibril aerogel described by one of the above-cited references might be manufactured to have a shaped volume via one of the techniques described, whereafter the shape is cut or otherwise subdivided into smaller, continuous shaped components of an absorbent core, e.g., a continuous homogeneous aerogel layer component. It is further contemplated that the aerogel might be manufactured and cast, e.g., in sheet form, and thereafter cut or otherwise divided to form such layer components.

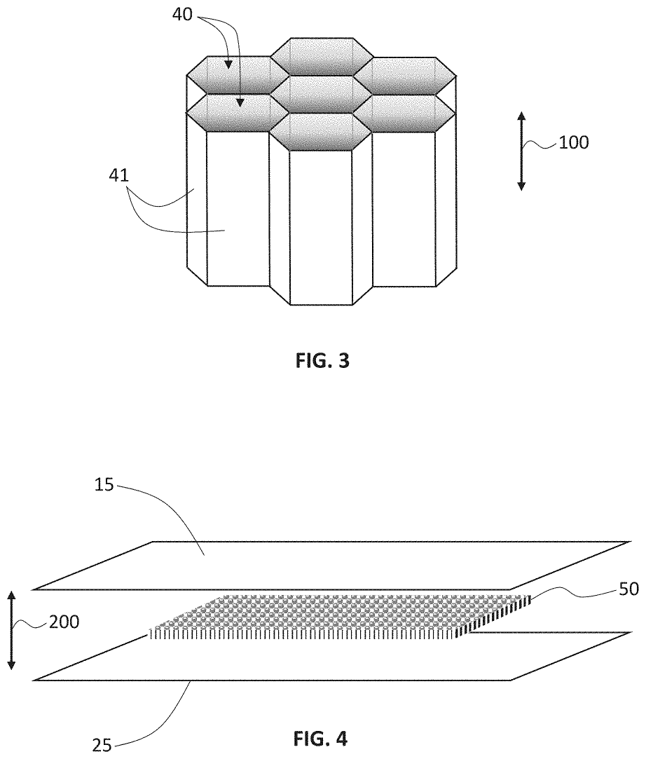

As described in the accompanying publications, a cellulose nanofibril aerogel might be produced via a controlled freeze and freeze-drying, or cyclic freezing and thawing (which may be repeated) followed by freeze-drying to produce isotropic or anisotropic pore morphology. Referring to FIG. 3, an aerogel may be produced with an anisotropic pore morphology that roughly resembles a honeycomb with walls defining elongate and interconnected pores 40, in which the walls 41 are generally, approximately and predominately aligned in planes lying along a particular direction 100, reflecting a more ordered formation with extended and more well-defined pore walls. Without intending to be bound by theory, it is believed that such anisotropic pore morphology may be preferred in a cellulose nanofibril aerogel to be used an absorbent component in an absorbent article, due to relatively higher absorbent capacity and relatively greater mechanical robustness in both dry and wet conditions.

Referring to FIG. 4, when a body of such an aerogel is included as a unitary, continuous layer component 50 in the absorbent structure of an absorbent article, it might be desired in some circumstances that the anisotropic pore morphology be such that the walls defining the pores be oriented predominately transversely or orthogonally (i.e., approximately, predominately aligned with a z-direction 200) to the wearer-facing surfaces of outer components such as a topsheet 15. Without intending to be bound by theory, it is believed that this orientation allows for more effective controlled absorption and containment of aqueous body fluids. The predominantly z-direction orientation of pore walls promotes aqueous liquid movement along the hydrophilic pore walls in the z-direction, such that pores tend to be filled completely proximate a fluid discharge location at the user's body, before absorbed fluid overflows into neighboring pores along the x- and y-directions. Conversely, an orientation in which the pore walls extend substantially along x- and y-directions can promote undesirable fluid flow along hydrophilic pore wall surfaces along x and/or y-directions, away from the point of fluid discharge and outwardly along the x- and/or y-directions toward the edges of the layer. This could ultimately lead to larger top stains and side soiling.

All documents cited in the Description of Embodiments are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments have been illustrated and described, it would be obvious to those skilled in the art that various other changes and modifications may be made without departing from the spirit and scope of the invention(s) described herein.

In view of the foregoing description, the following non-limiting examples of articles and methods with the combinations of features and steps recited are contemplated:

1. A disposable absorbent article, comprising a liquid permeable topsheet, a liquid-impermeable backsheet, and an absorbent core structure disposed between the topsheet and the backsheet, wherein the absorbent core structure comprises a cellulose aerogel body.

2. The disposable absorbent article of example 1 wherein the cellulose aerogel body comprises cellulose nanofibrils.

3. The disposable absorbent article of either of examples 1 or 2 wherein the cellulose aerogel of the body is derived from rice straw cellulose.

4. The disposable absorbent article of any of the preceding examples wherein the cellulose aerogel body has an anisotropic pore morphology.

5. The disposable absorbent article of any of the preceding examples wherein the absorbent core structure comprises a continuous layer formed by the aerogel body.

6. The disposable absorbent article of example 5 wherein the aerogel body forming the continuous layer has an anisotropic pore morphology with pore walls aligned predominately, approximately in a z-direction.

7. The disposable absorbent article of any of examples 1-6 wherein the absorbent structure comprises a plurality of discrete cellulose aerogel bodies.

8. The disposable absorbent article of example 7 wherein the discrete bodies are distributed within a matrix comprising filaments, fibers and/or foam.

9. The disposable absorbent article of example 8 wherein the filaments or fibers comprise filaments or fibers spun from a polymeric resin.

10. The disposable absorbent article of either of examples 8 or 9 wherein the filaments or fibers comprise cellulose fibers.

11. The disposable absorbent article of example 5 wherein the absorbent structure comprises a second continuous layer formed of a nonwoven web or batt of fibers or filaments, overlying the continuous aerogel layer to a wearer-facing side thereof.

12. The disposable absorbent article of example 11 wherein the filaments or fibers comprise filaments or fibers spun from a polymeric resin.

13. The disposable absorbent article of example 11 wherein the filaments or fibers comprise cellulose fibers.

14. The disposable absorbent article of any of the preceding examples wherein the absorbent core structure comprises a foam.

15. The disposable absorbent article of example 14 wherein the absorbent core structure comprises a layer comprising a continuous formation of the foam.

16. The disposable absorbent article of either of examples 14 or 15 wherein the foam is formed of a polymeric resin.

17. The disposable absorbent article of either of examples 14 or 15 wherein the foam is a HIPE foam.

18. The disposable absorbent article of any of the preceding examples wherein the cellulose aerogel body has a density as low as 8 grams per cubic centimeter and a porosity of at least 98.5%.

19. The disposable absorbent article of any of the preceding examples wherein the cellulose aerogel body has a distilled water absorbency of at least 104 grams water per gram dry aerogel body.

20. The disposable absorbent article of any of the preceding examples wherein the cellulose aerogel body has an acquisition rate of at least 25 grams per second per gram of solid aerogel when immersed in a distilled water bath.

21. The disposable absorbent article of any of the preceding examples wherein the cellulose aerogel body has a linear elastic compression modulus of at least 20 kPa when subjected to a compression test according to ASTM D 3575-91.

22. The disposable absorbent article of any of the preceding examples wherein the cellulose aerogel body has an elastic recovery of at least 80% in less than 10 s when subjected to a stress of 1 psi or lower testing according to ASTM D3574 Test M.

23. The disposable absorbent article of any of the preceding examples wherein the article is a feminine hygiene pad, an incontinence pad, a pair of incontinence pants, a diaper, a pair of training pants or a bandage or wound dressing product.

24. A method for manufacturing an absorbent structure of a disposable absorbent article, comprising the steps of:

providing a supply of cellulose nanofibrils;

preparing an aqueous colloidal suspension of the cellulose nanofibrils in a vessel, to form a hydrogel contained in the vessel;

removing water from the hydrogel;

forming a solid aerogel body; and

disposing the aerogel body or a portion thereof between a liquid permeable topsheet and a liquid-impermeable backsheet.

25. The method of example 24, comprising the additional steps of:

exchanging a substitute solvent for water in the hydrogel; and

removing substitute solvent from the suspension via drying under supercritical conditions.

26. The method of example 24, comprising the additional steps of:

freezing the hydrogel to form a frozen hydrogel; and

freeze-drying the frozen hydrogel.

27. The method of example 26, comprising the additional steps, after the freezing step and prior to the freeze-drying step, of:

thawing the frozen hydrogel; and

re-freezing the thawed hydrogel.

28. The method of either of examples 26 or 27 wherein or more freezing step/steps is/are performed in an environment having a temperature no lower than required to completely freeze the hydrogel within a period no less than 1 hour, more preferably no less than 2 hours, and even more preferably no less than 3 hours, and no greater than 15 hours.

29. The method of any of preceding examples 24 or 26-28, wherein the steps are controlled to impart anisotropic pore morphology to the aerogel body.

30. The method of any of examples 24-29, comprising the additional steps of:

dividing the aerogel body into aerogel particles; and

disposing the particles between the topsheet and the backsheet.

31. The method of example 30 comprising the additional steps of blending the aerogel particles with cellulose pulp fiber to form an absorbent blend, and disposing the absorbent blend between the topsheet and the backsheet.

32. The method of any of examples 24-29, comprising the additional steps of:

dividing the aerogel body into a plurality of aerogel body sections; and

disposing one or more of the aerogel body sections between the topsheet and the backsheet.

33. The method of any of examples 24-28 comprising disposing the hydrogel onto or into a casting body to form a hydrogel sheet prior to removal of water therefrom, and thereby casting the hydrogel to form a cast aerogel body having a sheet form.

34. The method of any of examples 24-33 wherein the cellulose nanofibrils are derived from plant fibers.

35. The method of example 34 wherein the plant fibers are obtained from rice straw.

36. The method of any of examples 24-35 wherein the article is a feminine hygiene pad, an incontinence pad, a pair of incontinence pants, a diaper, a pair of training pants or a bandage or wound dressing product.

What is claimed is:

1. A disposable absorbent article, comprising a liquid permeable topsheet, a liquid-impermeable backsheet, and an absorbent core structure disposed between the topsheet and the backsheet, wherein the absorbent core structure comprises a cellulose aerogel body.

2. The disposable absorbent article of claim 1 wherein the cellulose aerogel body comprises cellulose nanofibrils.

3. The disposable absorbent article of claim 1 wherein the cellulose aerogel of the body is derived from rice straw cellulose.

4. The disposable absorbent article of claim 1 wherein the cellulose aerogel body has an anisotropic pore morphology.

5. The disposable absorbent article of claim 1 wherein the absorbent core structure comprises a continuous layer formed by the aerogel body.

6. The disposable absorbent article of claim 5 wherein the aerogel body forming the continuous layer has an anisotropic pore morphology with pore walls aligned predominately, approximately in a z-direction.

7. The disposable absorbent article of claim 1 wherein the absorbent structure comprises a plurality of discrete cellulose aerogel bodies.

8. The disposable absorbent article of claim 7 wherein the discrete bodies are distributed within a matrix comprising filaments, fibers and/or foam.

9. The disposable absorbent article of claim 8 wherein the filaments or fibers comprise filaments or fibers spun from a polymeric resin.

10. The disposable absorbent article of claim 8 wherein the filaments or fibers comprise cellulose fibers.

11. The disposable absorbent article of claim 5 wherein the absorbent structure comprises a second continuous layer formed of a nonwoven web or batt of fibers or filaments, overlying the continuous aerogel layer to a wearer-facing side thereof.

12. The disposable absorbent article of claim 1 wherein the absorbent core structure comprises a foam.

13. The disposable absorbent article of claim 12 wherein the foam is a HIPE foam.

14. The disposable absorbent article of claim 1 wherein the article is a feminine hygiene pad, an incontinence pad, a pair of incontinence pants, a diaper, a pair of training pants or a bandage or wound dressing product.
